(19) United States
(12) Patent Application Publication
(10) Pub. No.: US 2007/0208293 A1
(43) Pub. Date: Sep. 6, 2007

(54) Methods and devices for noninvasive pressure measurment in ventricular shunts

(76) Inventors: Habah Noshy Mansour, La Mirada, CA (US); Ramez Emile Necola Shehada, La Mirada, CA (US)

Correspondence Address: HEBAH NOSHY MANSOUR, 14759 HARDAWAY DR., LA MIRADA, CA, 90638, US

(21) Appl. No.: 11/714,603

(22) Filed: Mar. 5, 2007

Related U.S. Application Data
(60) Provisional application No. 60/778,752, filed on Mar. 3, 2006.
(60) Provisional application No. 60/796,714, filed on May 2, 2006.

(51) Int. Cl. A61M 5/00

(52) U.S. Cl. 604/9; 604/8

(57) ABSTRACT

The present invention is directed to a system and method for monitoring the intraluminal pressure in a ventricular shunt. The shunt may have one or more measurement nodes housing a pressure-sensitive body that changes dimensions in response to the pressure of the cerebrospinal fluid within the lumen of the shunt. The change in the dimensions of the pressure-sensitive body may be measured transcutaneously using an ultrasonic transducer and processed using a processor to estimate the intraluminal pressure. The shunt may include one or more pressure-measurement nodes distributed along the length of the shunt to enable the detection of shunt occlusions and valve malfunctioning.

### CROSS-REFERENCE TO RELATED APPLICATION(S)

This application claims priority to U.S. Provisional Patent Application No. 60/778,752, filed Mar. 3, 2006, entitled Methods and devices for pressure measurement and infection reduction in neurosurgical shunts, and U.S. Provisional Patent Application No. 60/796,714, filed May 2, 2006, entitled Methods and devices for non-invasive pressure measurement in ventricular shunts, and incorporates the contents in their entirety.

### FIELD OF THE INVENTION

The present invention relates to a system and method for monitoring the pressure in a ventricular shunt, in particular, a system and method utilizing a pressure-sensitive body that changes shape in response to pressure within the lumen of the ventricular shunt.

### BACKGROUND OF THE INVENTION

Hydrocephalus is a condition in which the body is unable to relieve itself of excess cerebrospinal fluid collected in the ventricles of the brain because of infection or disease. The increase in the cerebrospinal fluid pressure may be caused by tumor of the brain or of the membranes covering the brain (e.g. meninges), infection of or bleeding into the cerebrospinal fluid, or congenital malformations of the brain.

Ventricular shunt is a surgical procedure in which a tube is placed in one of the brain ventricles to drain the excess cerebrospinal fluid and relieve the elevated pressure in hydrocephalus. The ventricular shunt drains fluid from the ventricular system in the brain to the cavity of the abdomen (e.g. peritoneal cavity) or to a large vein in the neck (e.g. the jugular vein).

The tubing contains unidirectional valves to insure that fluid can only flow out of the brain and not back into it. The valve can be set at a desired pressure to allow cerebrospinal fluid to escape whenever the pressure level is exceeded.

A small reservoir may be attached to the tubing and placed under the scalp. This reservoir allows samples of cerebrospinal fluid to be removed with a syringe and to check the pressure.

The pressure of the cerebrospinal fluid should be checked periodically to ensure that the pressure is relieved and the shunt is operating properly, and/or initiate a drug therapy if necessary. Therefore a means for the non-invasive measurement of the cerebrospinal fluid pressure along the length of the shunt is highly desirable to detect the degree and location of tube occlusion(s) and the malfunction of the valve(s).

### SUMMARY OF THE INVENTION

The current invention relates to a ventricular shunt (or shunt, used interchangeably herein) including a pressure-sensitive body that changes its dimensions in response to the pressure of the cerebrospinal fluid within the lumen of the shunt.

The change in the dimensions of the pressure-sensitive body may be measured transcutaneously using an ultrasonic transducer and processed using a processor to estimate the intraluminal pressure at the location of the pressure-sensitive body.

The pressure-sensitive body may be housed in a pressure-measurement node configured to ensure the proper aiming of the ultrasonic transducer on the pressure-sensitive body to improve the accuracy by which the dimensions of the pressure-sensitive body are measured.

The shunt may include one or more pressure-measurement nodes distributed along the length of the shunt to enable the detection of occluded shunt segments and/or the detection malfunctioning valves.

### BRIEF DESCRIPTION OF THE DRAWINGS

FIG. 1 shows an embodiment of a ventricular shunt and a monitoring unit in accordance with the present invention.

FIG. 2A shows a front view of the shunt of FIG. 1.

FIG. 2B shows a side view of the shunt of FIG. 1.

FIG. 2C shows a front view of a measurement node of the shunt of FIG. 1.

FIG. 2D shows a side view of a measurement node of the shunt of FIG. 1.

FIG. 3 shows an embodiment of an ultrasound probe positioned on the measurement node of the shunt of FIG. 1.

FIG. 4 shows another embodiment of the ultrasound probe positioned on an embodiment the measurement node of the shunt of FIG. 1.

FIG. 5A shows a front view of another embodiment of the shunt.

FIG. 5B shows a side view of another embodiment of the shunt.

FIG. 6A shows a front view of another embodiment of the measurement node.

FIG. 6B shows a side view of another embodiment of the measurement node.

FIG. 7A shows a front view of another embodiment of the measurement node.

FIG. 7B shows a side view of another embodiment of the measurement node.

FIG. 8A shows a front view of another embodiment of the shunt.

FIG. 8B shows a side view of another embodiment of the shunt.

FIG. 9 shows a front view of another embodiment of the shunt.

### DETAILED DESCRIPTION OF THE INVENTION

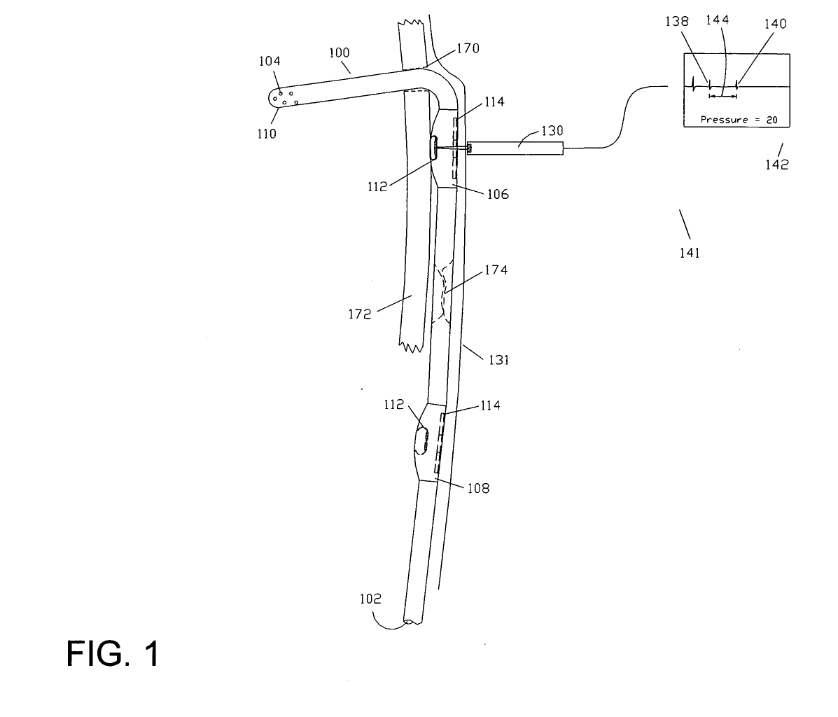

A shunt monitoring system is shown in FIG. 1. The shunt monitoring system may be comprised of a shunt 100 with measurement nodes 106 and 108 and a monitoring unit 141. The monitoring unit 141 may be comprised of a transducer 130 and a processor 142.

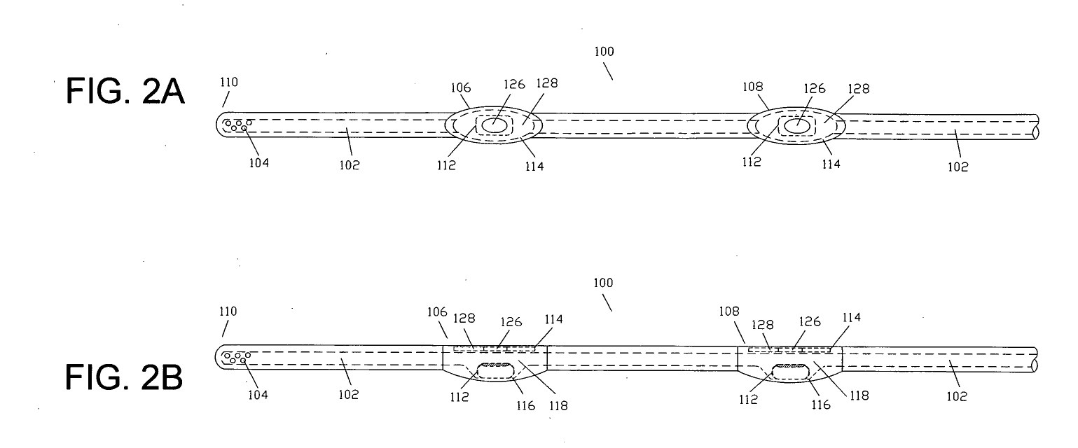

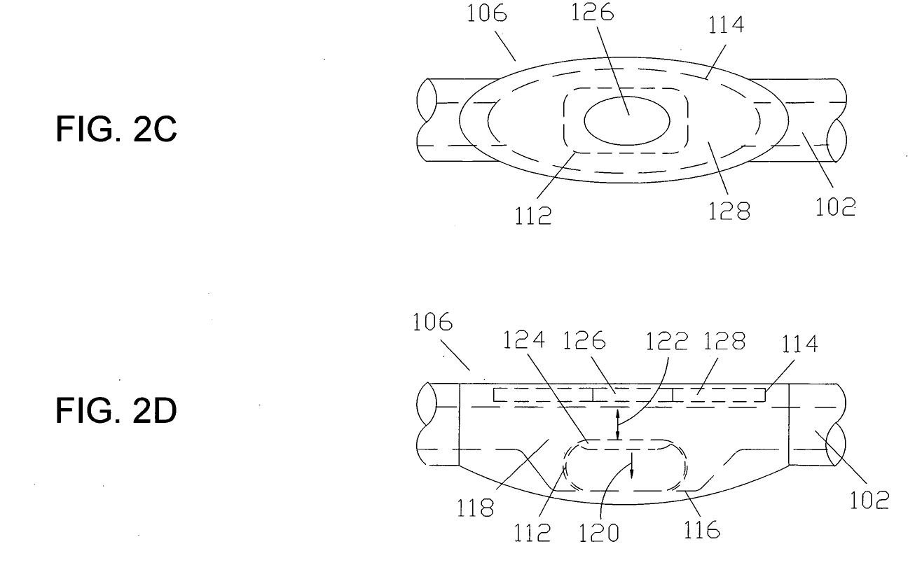

The preferred embodiment of the shunt 100 shown in FIG. 1 and FIG. 2 may be a flexible tube with a lumen 102, a set of draining holes 104, and at least two measurement nodes 106 and 108 positioned along the length of the shunt 100. The set of draining holes 104 may be located at the distal end 110 (or ventricular end, used interchangeably herein) of the shunt 100.

The nodes 106 and 108 may be preferably positioned along the length of the shunt 100 at locations that are accessible for interrogation by the external ultrasonic transducer 130 once the shunt 100 is placed inside the patient's body. The pressure measured at the node 106 closest to the ventricular end 110 may indicate the ventricular pressure, while a pressure difference between the nodes 106 and 108 may indicate an occlusion 174 between the locations of the nodes 106 and 108. Although, the above example describes a shunt with the two measurement nodes 106 and 108, additional measurement nodes may be used to enable the noninvasive detection of occlusion between additional segments of the shunt 100.

FIG. 2C and 2D show the details of the measurement node 106 (or 108) which may include a pressure-sensitive body 112 and an acoustic collimator 114. The pressure-sensitive body 112 may be a gas-filled capsule made of a flexible material such as silicone. The body 112 may be attached to the bottom 116 of the cavity 118 of the measurement node 106 (or 108). The cavity 118 may be continuous with the lumen 102. The body 112 may collapse in the direction 120 under increased pressure in the cavity 118. The wall thickness of the body 112 and/or the pressure of the gas within the body 112 may be selected to allow a predetermined degree of collapse of the body 112 under a given increase in the pressure within the cavity 118. Therefore the distance 122 between the upper surface 124 of the body 112 and the acoustically translucent window 126 of the collimator 114 will vary with the pressure within the cavity 118. A calibration relationship between the distance 122 and the pressure within the cavity 118 may be established and used by the processor 142 for the estimation of the pressure within the cavity 118 from a measured distance 122.

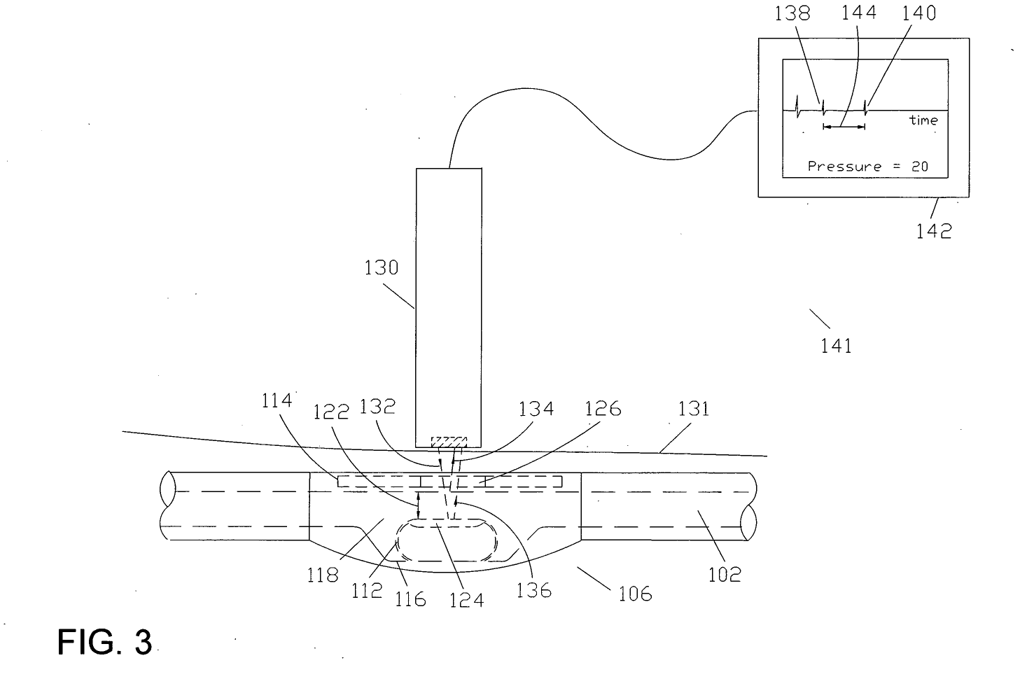

The distance 122 may be measured using an external ultrasound transducer 130 placed over the skin 131 covering the node 106 (or 108). The transducer 130 may emit ultrasound pulses 132 aimed towards the collimator 114 as shown in FIG. 3. The transducer 130 may receive the ultrasound echoes 134 and 136 returning from the acoustically translucent window 126 and the upper surface 124 of the body 112 and converts them into the electronic pulses 138 and 140, respectively. The electronic pulses 138 and 140 are processed by the processor 142 to determine the time difference 144 between their corresponding echoes 134 and 136. The time difference 144 between the ultrasound echoes 134 and 136 or their corresponding electronic pulses 138 and 140 is indicative of the distance 122. The processor 142 may convert the time difference 144 into the value of the pressure within the node 106 (or 108) using the established calibration relationship.

The acoustical collimator 114 may be composed of an acoustically translucent window 126 surrounded by an acoustically opaque annulus 128 as shown in FIGS. 2C and 2D. The annulus 128 may be made of an acoustically opaque material such as, for example, TYVEK polymer (commercially available from DuPont), a foamy material, or a gas-filled volume to provide high acoustical attenuation. The collimator 114 may be positioned such that its acoustically translucent window 126 is directly above the upper surface 124 of the body 112. Therefore, the collimator 114 may only allow the passage of ultrasonic pulses 132 that are traveling approximately perpendicular to the upper surface 124, which may improve the accuracy and repeatability of the measurement of the distance 122. In addition, this configuration may eliminate the guesswork re the proper aiming of the transducer 130 and ensure that the returned ultrasound echoes 134 and 136 are arising from the pressure-sensitive body 112. The body of the node 106 (or 108) may be made of rigid and/or semi-flexible materials necessary to maintain the dimensions of the cavity 118.

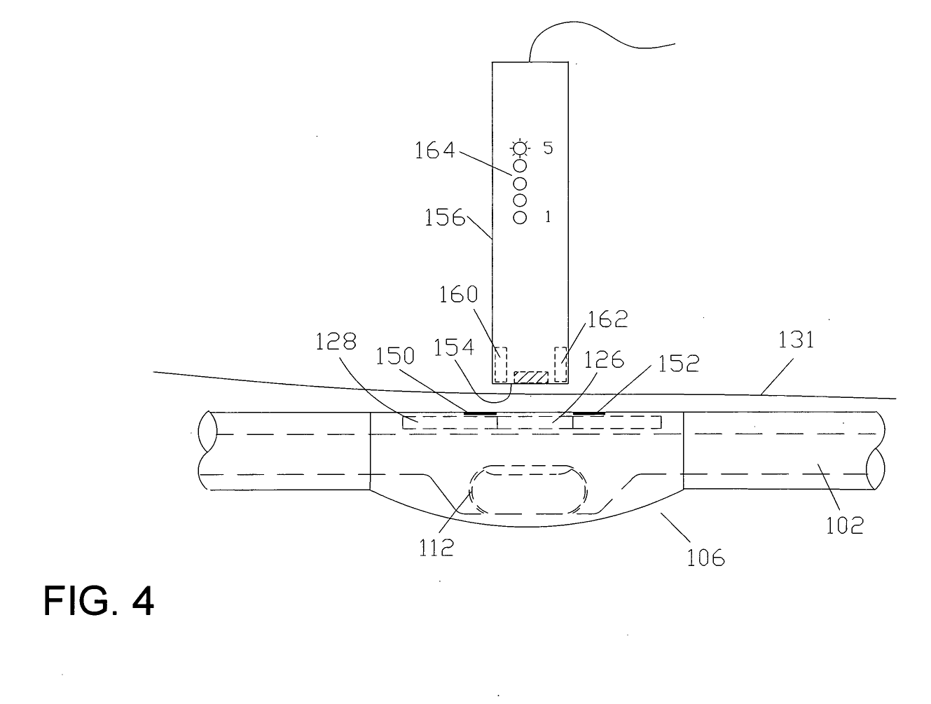

Another embodiment of the measurement node 106 (or 108) shown in FIG. 4 includes metallic or magnetic bodies 150 and 152 to help guide the operator to the location of the node 106 (or 108) under the skin 131 without having to palpate. The sensing tip 154 of the ultrasound transducer 156 may be equipped with metal and/or magnetic sensors 160 and 162 that may activate an audio tone or a light indicator 164 once the tip 154 is adjacent to the measurement node 106 (or 108). For example, a signal strength indicator 164 (e.g. an optical bar graph) may be used to indicate whenever the tip 154 is over the node 106 (or 108) and aligned with the acoustical window 126.

In a typical application of the shunt 100, the distal end 110 of the shunt 100 may be inserted through a hole 170 in the skull 172 into the frontal horn of the right ventricle as shown in FIG. 1. The shunt 100 may be tunnelized under the skin 131 all the way down to the peritoneal cavity or to a major vein where it drains. The shunt 100 is positioned such that the collimator 114 of the nodes 106 and 108 is facing the skin 131 as shown in FIG. 1 and FIG. 3 to enable the easy interrogation of the pressure-sensing body 112 by the ultrasound transducer 130 placed over the skin 131. The pressure measured from the node 106 positioned close to the ventricular end 110 may represent the intraventricular pressure. A difference in the pressure between the node 106 and the node 108 may indicate the presence of an occlusion 174 between the two nodes. The magnitude of the difference in the pressure between the node 106 and the node 108 may indicate the degree of the occlusion 174 between the two nodes. The occlusion 174 may lead to an increase in the pressure measured at node 106 relative to the pressure measured at node 108. An equivalent increase in the pressure at both nodes 108 and 106 above the opening pressure of the shunt's valve may be indicative of valve malfunctioning.

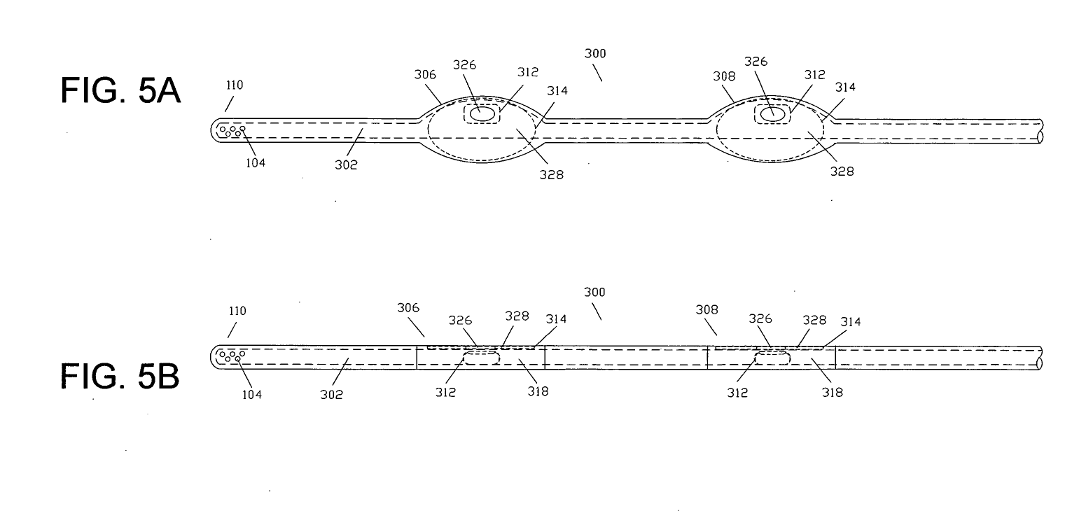

Another measurement node embodiment is shown in FIG. 5 where the measurement node 306 (or 308) is located on the side of the shunt 300. The cavity 318 is opened directly to the lumen 302. This configuration may have several advantages including that the pressure-sensitive body 312 is positioned away from the lumen 302 and hence it does not affect the effective diameter of the lumen 302. The winged geometry of the node 306 (or 308) with respect to the shunt 300 ensures the proper orientation of the node 306 (or 308) relative to the surface of the skin where the ultrasound transducer 130 may be placed to interrogate the pressure-sensitive body 312. In addition, having the node 308 on the side of the shunt 300 does not increase the overall thickness of the shunt 300 while still making it easier to palpate the location of the node 306 (or 308) through the skin. The collimator 314 may be elliptical in shape with an off-centered acoustical window 326 surrounded by the acoustically opaque shield 328.

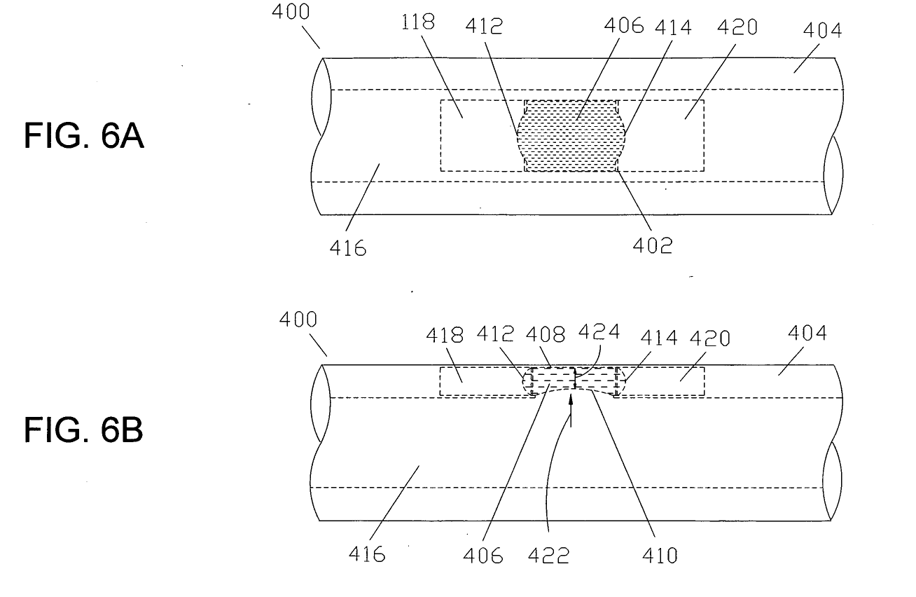

Another embodiment of the measurement node is shown in FIG. 6 where the pressure-sensitive body 402 may be embedded within the wall 404 of the shunt 400. In this embodiment the pressure-sensitive body 402 may be a hollow rectangular prism filled with biologically compatible fluid 406 such as for example saline. The upper wall 408 of the body 402 may be non-deformable while the lower wall 410 and the sidewalls 412 and 414 may be expansible diaphragms made of a flexible material such as silicone. The lower wall 410 may be bordering the lumen 416 of the shunt 400. The sidewalls 412 and 414 may be bordering the gas-filled chambers 418 and 420, respectively. An increase in the pressure within the lumen 416 may press against the lower wall 410 to displace it in the direction 422 and decrease the distance 424 between the lower wall 410 and the upper wall 408. Simultaneously, the flexible sidewalls 412 and 414 may bulge into the bordering gas-filled chambers 418 and 420 to relieve the fluid volume shifted by the displacement of the lower wall 410. This configuration may reduce the resistance to fluid shifts and therefore maximize the displacement of the lower wall 410 in response to a given increase in the intraluminal pressure.

Similar to the other embodiments, the distance 424 may be measured transcutaneously using the ultrasonic transducer 130 and used by the processor 142 to estimate the pressure within the lumen 416.

The lower wall 410 may be embedded with micro air or gas bubbles (not shown) to increase its ultrasonic contrast relative to the fluid 406 and improve its detection using the external ultrasound transducer 130. In addition, ultrasonic contrast and detection of the lower wall 410 may be also improved by using a fluid 406 with acoustical characteristics that are different from that of the material of the diaphragm 410 and/or that of the biological fluid that would be normally filling the lumen 416 (i.e. the cerebrospinal fluid). The presence of air (or gas) in the chambers 418 and 420 may act as an acoustical collimator that would only allow the passage of ultrasonic waves that are almost perpendicular to both the upper wall 408 and the lower wall 410.

Although the above embodiment describes a pressure sensitive body that is rectangular in shape, the body may assume any other shape including cylindrical. A cylindrical pressure-sensitive body (not shown) may be encircled all-around by a gas-filled compartment to allow the expansion of the cylinder's flexible wall into the surrounding compartment and hence facilitate the movement of the cylinder's flexible bottom (or diaphragm) in response to an increase in the intraluminal pressure.

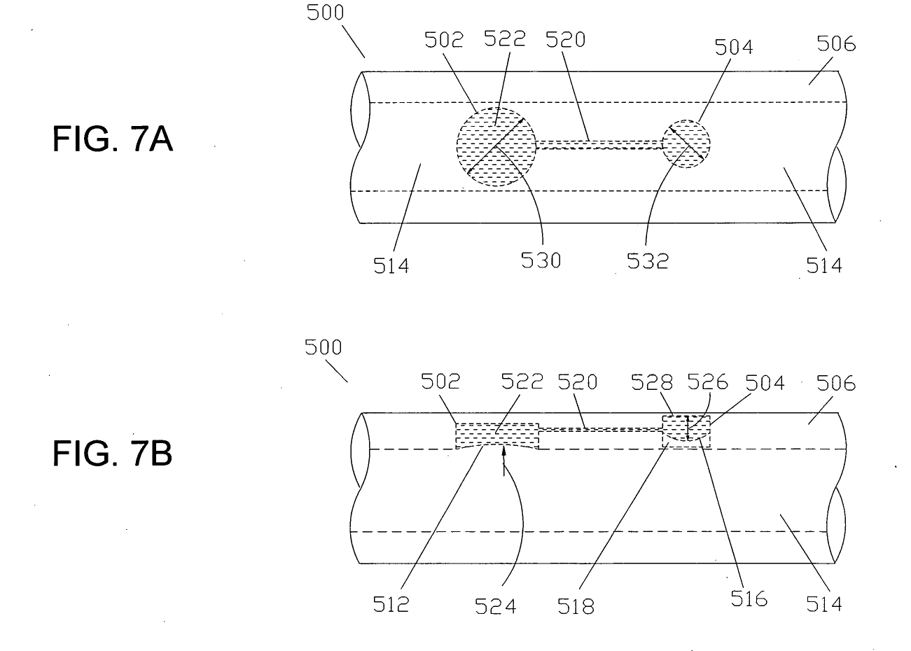

Another embodiment of the measurement node is shown in FIG. 7 where the pressure-sensitive body may be composed of at least the two hydraulically connected chambers 502 and 504 embedded within the wall 506 of the shunt 500. The pressure-sensing chamber 502 may be composed of non-expansible (e.g. rigid) walls except for a flexible diaphragm 512 bordering the lumen 514 of the shunt 500. The pressure-reading chamber 504 may be composed of non-expansible (e.g. rigid) walls except for a diaphragm 516 bordering a gas-filled compartment 518. The chambers 502 and 504 may be interconnected with a non-expansible channel 520 and filled with a fluid 522. The fluid 522 may hydrostatically transmit the pressure signals from the sensing chamber 502 to the reading chamber 504.

An increase in the pressure within the lumen 514 may press against the diaphragm bottom 512 to displace it in the direction 524 and shift some of the fluid 522 into the chamber 504. The shift of the fluid 522 into the chamber 504 may displace the diaphragm 516 into the gas-filled compartment 518 to increase the distance 526 between the diaphragm 516 and the top wall 528. A calibration relationship between the distance 526 and the pressure may be established and used for the estimation of the pressure from a measured distance 526.

Similar to the other embodiments, the distance 526 may be measured transcutaneously using the ultrasonic transducer 130 and used by the processor 142 to estimate the pressure within the lumen 514.

This embodiment may allow the amplification of the displacement of the sensing diaphragm 512 into a larger displacement of the reading diaphragm 516 by making the diameter 530 of the pressure-sensing chamber 502 greater than the diameter 532 of the pressure-reading chamber 504. This may be helpful in detecting slight changes in pressure and in improving the accuracy of the pressure estimation from diaphragm displacement.

This embodiment may also allow the placement of the sensing chamber 502 at a distant location from the reading chamber 504. For example, the sensing chamber 502 may be placed near the ventricular end of the shunt that is inside the skull (inaccessible to ultrasonic interrogation), while the reading chamber 504 may be placed at a location that is outside the skull and is accessible for ultrasonic measurements.

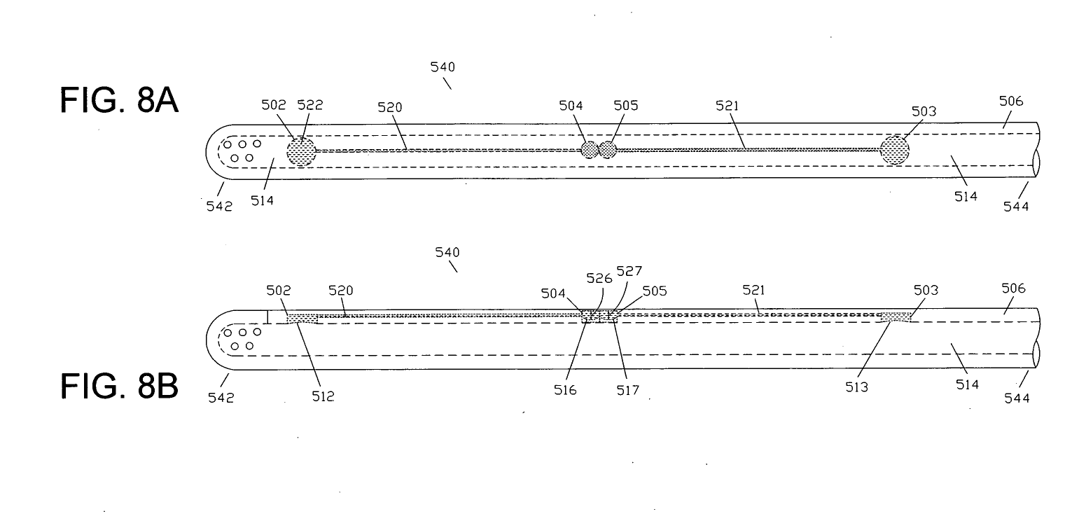

This embodiment may also enable the sensing of the intraluminal pressure at multiple locations along the length of the shunt while allowing the reading of all these measurements at a single location that is transcutaneously accessible by ultrasonic means. For example, the shunt embodiment 540 shown in FIG. 8 include a first sensing-chamber 502 that is located near to the ventricular end 542 of the shunt 540 and a second sensing-chamber 503 that is located near the proximal end 544 of the shunt 540. Both sensing-chambers 502 and 503 are hydraulically connected through the fluid-filled channels 520 and 521 to the adjacent reading-chambers 504 and 505, respectively. Relative difference in the expansion distances 526 and 527 of the diaphragms 516 and 517 measured at the reading-chambers 504 and 505 may be indicative of pressure differences or an occlusion between the locations of the sensing-chambers 502 and 503, respectively. An ultrasound array transducer (not shown) operating in B-mode may be used to image the reading chambers 504 and 505 simultaneously and measure the distances 526 and 527.

The distances 526 and 527 may be processed by a processor to estimate pressure at the locations of the sensing chambers 502 and 503 and the estimated pressures may be compared to detect the presence of an occlusion between sensing chambers 502 and 503. Although the above example only describes two sensing-chambers reporting to two adjacent reading-chambers, it is understood that additional reading and sensing chambers may be added to this configuration to enable the monitoring of pressure and occlusion at additional locations along the length of the shunt.

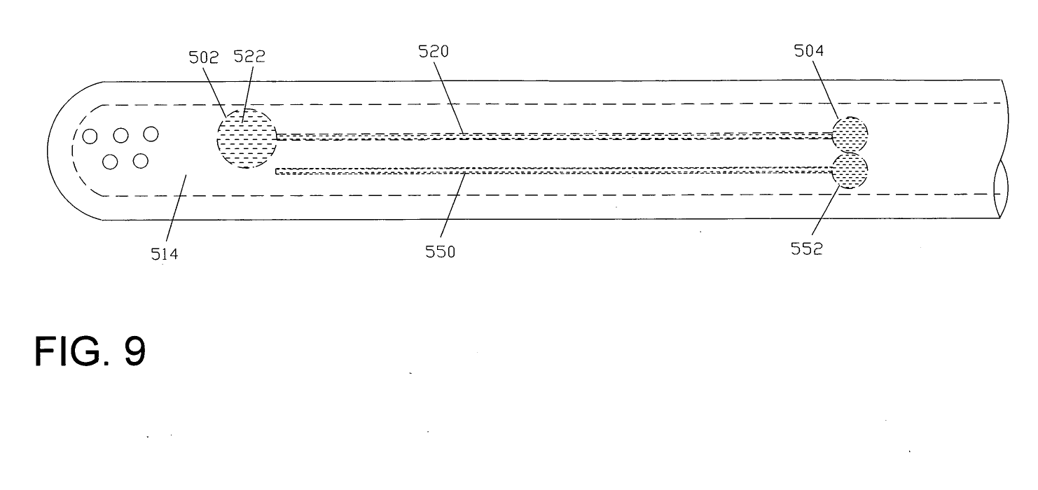

During the placement of the shunt 540 in the patient, the bending of the shunt 540 may cause the fluid 522 to shift within, for example, the channel 520 and cause an initial offset of the diaphragm 516. This offset may be corrected by recording the initial distance 526 and use it as baseline for comparison to future measurements of the distance 526. Alternatively, the offset may be eliminated or minimized by using a second dummy channel 550 (i.e. not connected to a pressure-sensing chamber) running next to the channel 520 and connected to a second reading-chamber 552 as shown in FIG. 9. Therefore, the diaphragm displacement of the second reading chamber 552 will be solely caused by pressure-unrelated factors such as the mechanical bending of the shunt 540 and/or abnormal temperature changes. The difference in the displacement distances between the diaphragms of the first and second pressure-reading chambers 504 and 552 may be considered as the actual displacement distance caused by the pressure variation at the location of the pressure-sensing chamber 502.

Although the above detailed description describes and illustrates various preferred embodiments, the invention is not so limited. Many modifications and variations will now occur to persons skilled in the art. As such, the preceding description has been presented with reference to presently preferred embodiments of the invention. Workers skilled in the art and technology to which this invention pertains will appreciate that alterations and changes in the described structure may be practiced without meaningfully departing from the principal, spirit and scope of this invention.

Accordingly, the foregoing description should not be read as pertaining only to the precise structures described and illustrated in the accompanying drawings, but rather should be read consistent with and as support to the following claims which are to have their fullest and fair scope.

What is claimed is:

1. A ventricular shunt for draining a cerebrospinal fluid from a brain comprising:
a tubing with a lumen;
a first node disposed at a first location along the length of the tubing;
the first node includes a first body that is configured to change dimensions in response to a first pressure at the first location;

2. The shunt of claim 1, wherein the dimensions of the first body is measured ultrasonically.

3. The shunt of claim 1, wherein the dimensions of the first body are used to determine the first pressure at the first location.

4. The shunt of claim 1, wherein the first pressure is determined from the dimensions of the first body using a predetermined calibration.

5. A ventricular shunt for draining a cerebrospinal fluid from a brain comprising:
a tubing with a lumen;
a first node disposed at a first location along the length of the tubing;
a second node disposed at a second location along the length of the tubing;
the first node includes a first body that is configured to change dimensions in response to a first pressure at the first location;
the second node includes a second body that is configured to change dimensions in response to a second pressure at the second location;

6. The shunt of claim 5, wherein the dimensions of the first body and the dimensions of the second body are measured ultrasonically.

7. The shunt of claim 5, wherein the dimensions of the first body are used to determine the first pressure and the dimensions of the second body are used to determine the second pressure.

8. The shunt of claim 5, wherein a difference between the first pressure and the second pressure is used to detect occlusion between the first location and the second location.
